(19) United States
(12) Patent Application Publication
(10) Pub. No.: US 2026/0112501 A1
(43) Pub. Date: Apr. 23, 2026

(54) GAMIFICATION-BASED SMART WEIGHT MANAGEMENT DEVICE

(71) Applicant: Baekburn Inc, Chuncheon-si (KR)

(72) Inventor: TAEHOON YOON, Seoul (KR)

(73) Assignee: Baekburn Inc, Chuncheon-si (KR)

(21) Appl. No.: 19/359,073

(22) Filed: Oct. 15, 2025

(30) Foreign Application Priority Data
Oct. 17, 2024 (KR) 10-2024-0142263

(51) Int. Cl. G16H 50/30; A61B 5/00; A63B 24/00

(52) U.S. Cl. CPC G16H 50/30; A61B 5/4872; A63B 24/0075; A63B 2230/705

(57) ABSTRACT

A gamification-based smart weight management device, includes a next-day mission generation unit generating a mission for victory of a next day on a user terminal; a lifestyle pattern data collection unit collecting lifestyle pattern data of the user through the user terminal; a victory achievement prediction unit analyzing the lifestyle pattern data to determine a past day with a most similar lifestyle pattern data and predicts whether the victory is achieved based on a lifestyle pattern data from the past day; a mission performance detection unit analyzing the lifestyle pattern data to determine whether the mission is performed when the victory is predicted; an action plan change unit changing at least one action plan based on lifestyle pattern data; and a user level determination unit analyzing whether the victory or the mission is achieved and adjusting the game level of the user or the at least one action plan.

### CROSS-REFERENCE TO RELATED APPLICATION

This application claims priority to Korean Patent Application No. 10-2024-0142263 (filed on Oct. 17, 2024), which is hereby incorporated herein by reference in its entirety.

### BACKGROUND

The present disclosure relates to a weight management technology, and more particularly, to a gamification-based smart weight management device that can set a standard weight loss goal for the next day based on a game level of a user and adjust an action plan based on the prediction of achievement of the standard weight loss goal.

Recently, due to the influence of Westernized eating habits, overweight and obesity rates have been rapidly increasing, raising concerns about the increase in various adult diseases. According to the Journal of the American Medical Association, approximately 60% of Americans suffer from overweight or obesity, and in Korea, approximately 26% of adults are known to suffer from overweight. Furthermore, the prevalence of childhood obesity in Korea has reached approximately 20%, a figure that has doubled compared to three years ago. This obesity is a major cause of diseases such as diabetes and high blood pressure, and over 80% of childhood obesity leads to adult obesity. Therefore, there is an urgent need for various health management concepts, including diet, in society.

Reflecting this reality, interest in exercise is growing across society, and the general public's interest in dieting continues to grow due to obesity and overweight. Consequently, weight management technologies such as calorie counters, diet management apps, and exercise tracking devices are being developed. In response to this trend, various companies and research institutes both domestically and internationally are developing and commercializing equipment and programs that measure physical fitness and health through exercise.

However, existing systems have the disadvantage of focusing on measuring and analyzing exercise status. Furthermore, most people, even after making ambitious exercise plans, fail to stick to them and often give up midway. The actual effects of exercise can only be achieved through continuous and consistent practice. To achieve this, motivation and interest should be generated during exercise. Furthermore, the conventional weight management technologies fail to adequately reflect a lifestyle pattern and characteristics of a user, making it difficult to immediately adjust exercise plans based on changing circumstances of the user.

Korean Patent No. 10-0525773 relates to a weight-based health management method and a recording medium thereof, and includes a step of creating a database by inputting the weight and exercise amount of each individual, a step of calculating a weight gain/loss breakpoint exercise amount using the created database, and by using the calculated weight gain/loss breakpoint exercise amount, suggesting an exercise amount that is lower than the weight gain/loss breakpoint exercise amount when the user wants to gain weight, suggesting maintaining the weight gain/loss breakpoint exercise amount when the user wants to maintain the current weight, and suggesting an exercise amount that is higher than the weight gain/loss breakpoint exercise amount when the user wants to lose weight.

### SUMMARY

One embodiment of the present disclosure provides a gamification-based smart weight management device that can collect lifestyle pattern data of a user and predict whether a mission is achieved based on the lifestyle pattern data.

One embodiment of the present disclosure provides a gamification-based smart weight management device that can change an action plan based on past lifestyle pattern data of the user when it is difficult to achieve a mission.

One embodiment of the present disclosure provides a gamification-based smart weight management device that can adjust a game level or action plan of the user depending on whether a mission is achieved.

According to embodiments, there is provided a gamification-based smart weight management device including: a next-day mission generation unit that generates a mission for victory of a next day on a user terminal, the victory representing a baseline weight loss for the next day and the mission being determined based on a game level of a user and including at least one action plan; a lifestyle pattern data collection unit that collects lifestyle pattern data of the user through the user terminal; a victory achievement prediction unit that analyzes the lifestyle pattern data of the user to determine a past day with a most similar lifestyle pattern data and predicts whether the victory is achieved based on a lifestyle pattern data from the past day; a mission performance detection unit that analyzes the lifestyle pattern data of the user to determine whether the mission is performed when the victory is predicted; an action plan change unit that changes the at least one action plan based on lifestyle pattern data of the past day when the victory is not predicted; and a user level determination unit that analyzes whether the victory or the mission is achieved and adjusts the game level of the user or the at least one action plan.

The next-day mission generation unit may determine the baseline weight loss through an artificial intelligence weight loss model that trains an exercise amount of the user as input data and a weight change of the user as output data.

The next-day mission generation unit may provide usual behavioral data of the user to an artificial intelligence action plan model to determine a behavioral pattern of the user equal to or more than a certain standard frequency and determine the at least one action plan based on the behavioral pattern.

The next-day mission generation unit may analyze daily behavior data from the usual behavior data through the artificial intelligence action plan model, assign attention between the daily behavior data to determine a major behavior pattern, virtually generate a major action as the major behavior pattern, and determine the most similar N (where N is a natural number) action plans among all action plans as at least one action plan.

The victory achievement prediction unit may determine past population based on a location of the user, extracts time-based behavioral feature data based on the lifestyle pattern data of the user, and determine the past day from the past population.

The mission performance detection unit may additionally provide the user terminal with a mission that can be executed in the remaining day when a predicted probability of achieving the victory gradually decreases.

The action plan change unit may present lifestyle habit content required in the future or a degree of increase in an action plan required in the future based on the lifestyle pattern data of the past day, through the user terminal.

The user level determination unit may determine level up or level down based on a mission achievement win rate, victory achievement win rate, and weight change between the same day and the next day of the user.

The disclosed technology may have the following effects. However, this does not mean that a particular embodiment must include all or only the following effects, and therefore the scope of the disclosed technology should not be construed as being limited thereby.

The gamification-based smart weight management device according to one embodiment of the present disclosure can collect the lifestyle pattern data of the user and predict whether the mission is achieved based on the lifestyle pattern data.

The gamification-based smart weight management device according to one embodiment of the present disclosure can change the action plan based on the past lifestyle pattern data of the user when it is difficult to achieve the mission.

The gamification-based smart weight management device according to one embodiment of the present disclosure can adjust the game level or action plan of the user depending on whether the mission is achieved.

### BRIEF DESCRIPTION OF THE DRAWINGS

FIG. 1 is a diagram illustrating a smart weight management system according to the present disclosure.

FIG. 2 is a diagram illustrating a system configuration of a smart weight management device of FIG. 1.

FIG. 3 is a diagram illustrating a functional configuration of the smart weight management device of FIG. 1.

FIG. 4 is a flowchart illustrating one embodiment of a transformer-based electronic disclosure document service platform method according to the present disclosure.

FIG. 5 is a diagram illustrating a user level determination process according to mission performance according to one embodiment of the smart weight management device according to the present disclosure.

FIG. 6 is a diagram illustrating one embodiment of the smart weight management device according to the present disclosure.

### DETAILED DESCRIPTION

A description of the present disclosure is merely an embodiment for a structural or functional description and the scope of the present disclosure should not be construed as being limited by an embodiment described in a text. That is, since the embodiment can be variously changed and have various forms, the scope of the present disclosure should be understood to include equivalents capable of realizing the technical spirit. Further, it should be understood that since a specific embodiment should include all objects or effects or include only the effect, the scope of the present disclosure is limited by the object or effect.

Meanwhile, meanings of terms described in the present application should be understood as follows.

The terms “first,” “second,” and the like are used to differentiate a certain component from other components, but the scope of should not be construed to be limited by the terms. For example, a first component may be referred to as a second component, and similarly, the second component may be referred to as the first component.

It should be understood that, when it is described that a component is “connected to” another component, the component may be directly connected to another component or a third component may be present therebetween. In contrast, it should be understood that, when it is described that an element is “directly connected to” another element, it is understood that no element is present between the element and another element. Meanwhile, other expressions describing the relationship of the components, that is, expressions such as “between” and “directly between” or “adjacent to” and “directly adjacent to” should be similarly interpreted.

It is to be understood that the singular expression encompasses a plurality of expressions unless the context clearly dictates otherwise and it should be understood that term “include” or “have” indicates that a feature, a number, a step, an operation, a component, a part or the combination thereof described in the specification is present, but does not exclude a possibility of presence or addition of one or more other features, numbers, steps, operations, components, parts or combinations thereof, in advance.

In each step, reference numerals (e.g., a, b, c, etc.) are used for convenience of description, the reference numerals are not used to describe the order of the steps and unless otherwise stated, it may occur differently from the order specified. That is, the respective steps may be performed similarly to the specified order, performed substantially simultaneously, and performed in an opposite order.

The present disclosure can be implemented as a computer-readable code on a computer-readable recording medium and the computer-readable recording medium includes all types of recording devices for storing data that can be read by a computer system. Examples of the computer readable recording medium may include a ROM, a RAM, a CD-ROM, a magnetic tape, a floppy disk, an optical data storage device, and the like. Further, the computer readable recording media may be stored and executed as codes which may be distributed in the computer system connected through a network and read by a computer in a distribution method.

If it is not contrarily defined, all terms used herein have the same meanings as those generally understood by those skilled in the art. Terms which are defined in a generally used dictionary should be interpreted to have the same meanings as the meanings in the context of the related art, and are not interpreted as ideal meanings or excessively formal meanings unless clearly defined in the present application.

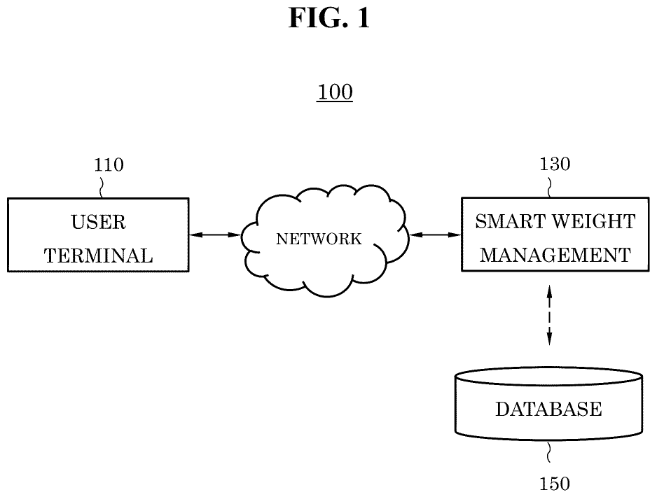

FIG. 1 is a diagram illustrating a smart weight management system according to the present disclosure.

Referring to FIG. 1, a smart weight management system 100 may include a user terminal 110, a smart weight management device 130, and a database 150.

The user terminal 110 may correspond to a terminal device operated by a user. In the embodiment of the present disclosure, the user may be understood as one or more users, and each of the one or more users may correspond to one or more user terminals 110. That is, although FIG. 1 illustrates one user terminal 110, a first user may correspond to a first user terminal, a second user may correspond to a second user terminal, and an nth user (where n is a natural number) may correspond to an nth user terminal, respectively.

In addition, the user terminal 110 may be implemented as one device constituting the smart weight management system 100 according to the present disclosure, and may be implemented in various forms depending on the company or institution that operates the health management service, fitness app, and diet management program.

In addition, the user terminal 110 may be implemented as a smart phone, laptop, or computer that is connected to and operable with a smart weight management device 130, but is not necessarily limited thereto and may also be implemented as various devices including tablet PCS, or the like.

Meanwhile, the user terminal 110 may be connected to the smart weight management device 130 via a network, and a plurality of user terminals 110 may be connected to the smart weight management device 130 simultaneously. The user terminal 110 may install and run a dedicated app (APP) for linking with the smart weight management device 130.

The smart weight management device 130 may be implemented as a computer or server that performs the gamification-based smart weight management method according to the present disclosure. Furthermore, the smart weight management device 130 may be connected to a user terminal 110 via a wired network or a wireless network such as Bluetooth, WiFi, or LTE, and may transmit and receive data with the user terminal 110 via the network.

Additionally, the smart weight management device 130 may be implemented to operate in connection with an independent external system (not illustrated in FIG. 1). For example, the smart weight management device 130 may operate in conjunction with a platform system that provides a smart weight management service.

The database 150 may correspond to a storage device that stores various information required during the operation of the smart weight management device 130. For example, the database 150 may store lifestyle pattern data of the user, including an exercise amount, meal time, sleep pattern, or the like of the user as well as missions provided to the user. However, the database 150 is not necessarily limited thereto, and may store information collected or processed in various forms during the process of the smart weight management device 130 providing a gamification-based smart weight management service according to the present disclosure.

In addition, in FIG. 1, the database 150 is depicted as a device independent of the smart weight management device 130, but it is not necessarily limited thereto, and the database 150 may be implemented as a logical storage device included in the smart weight management device 130.

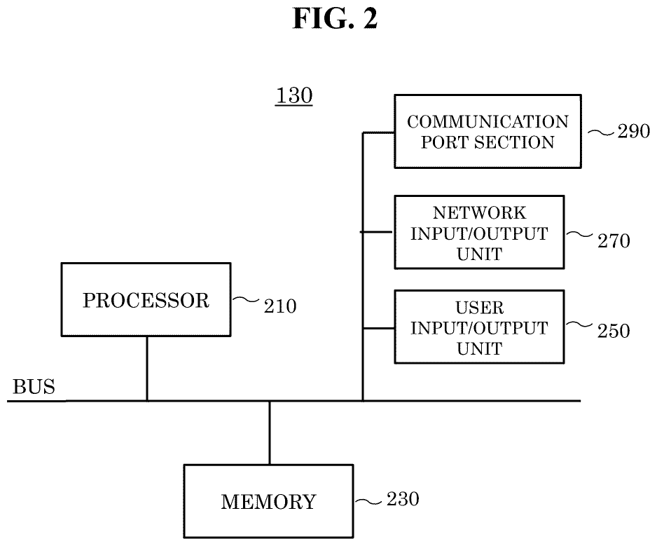

FIG. 2 is a diagram illustrating the system configuration of the smart weight management device of FIG. 1.

Referring to FIG. 2, the smart weight management device 130 may include a processor 210, memory 230, a user input/output unit 250, a network input/output unit 270, and a communication port unit 290.

The processor 210 may execute a gamification-based smart weight management procedure according to an embodiment of the present disclosure, manage the memory 230 that is read or written during this process, and schedule a synchronization time between the volatile memory and the non-volatile memory in the memory 230. The processor 210 may control the overall operation of the smart weight management device 130, and may be electrically connected to the memory 230, the user input/output unit 250, and the network input/output unit 270 to control the data flow therebetween. The processor 210 may be implemented as a Central Processing Unit (CPU) or a Graphics Processing Unit (GPU) of the smart weight management device 130.

The memory 230 may include an auxiliary memory device implemented as a non-volatile memory such as a Solid-State Disk (ISSD) or a Hard Disk Drive (HDD) and used to store all data required for the smart weight management device 130, and may include a main memory device implemented as a volatile memory such as a Random Access Memory (RAM). In addition, the memory 230 may store a set of commands for executing a gamification-based smart weight management method according to the present disclosure by being executed by the electrically connected processor 210.

The user input/output unit 250 includes an environment for receiving user input and an environment for outputting specific information to the user, and may include, for example, an input device including an adapter such as a touchpad, a touch screen, a virtual keyboard, or a pointing device, and an output device including an adapter such as a monitor or a touch screen. In one embodiment, the user input/output unit 250 may correspond to a computing device connected via remote access, and in such a case, the smart weight management device 130 may be performed as an independent server.

The network input/output unit 270 provides a communication environment for connecting to the user terminal 110 via a network, and may include, for example, an adapter for communication such as a Local Area Network (LAN), a Metropolitan Area Network (MAN), a Wide Area Network (WAN), and a Value-Added Network (VAN). In addition, the network input/output unit 270 may be implemented to provide a short-range communication function such as WiFi or Bluetooth, or a wireless communication function of 4G or higher for wireless transmission of data.

The communication port unit 290 may be implemented as a port mapping table that performs data routing during the process of transmitting and receiving data over a network. Here, the communication port unit 290 can distinguish communication sessions between the user terminal 110 and the server by assigning a unique source port to the user terminal 110, thereby preventing data collisions during the data transmission and reception process.

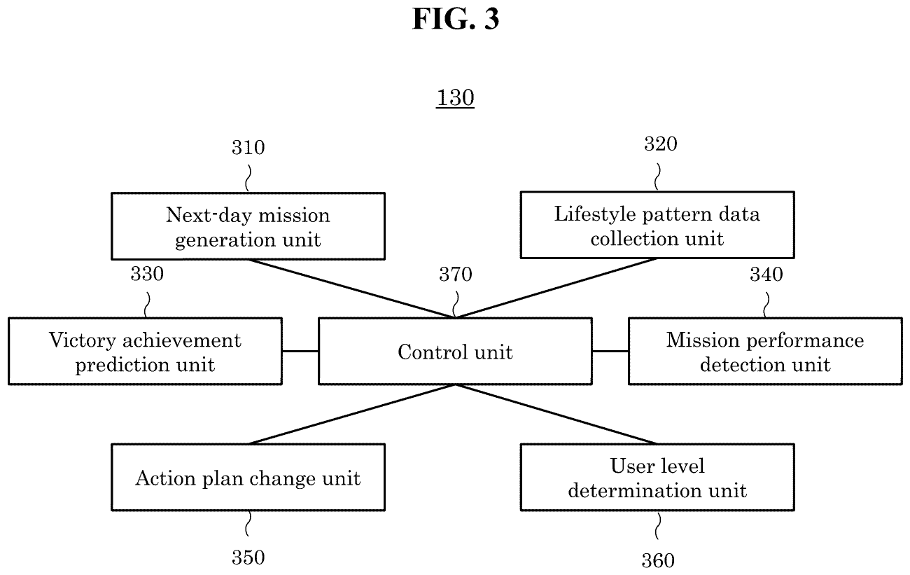

FIG. 3 is a diagram illustrating the functional configuration of the smart weight management device of FIG. 1.

Referring to FIG. 3, a smart weight management device 130 may perform a gamification-based smart weight management method according to the present disclosure. To this end, the smart weight management device 130 may include a next-day mission generation unit 310, a lifestyle pattern data collection unit 320, a victory achievement prediction unit 330, a mission performance detection unit 340, an action plan change unit 350, a user level determination unit 360, and a control unit 370.

In this case, the embodiments of the present disclosure do not necessarily include all of the above-described components simultaneously. Depending on each embodiment, some of the above-described components may be omitted, or some or all of the above-described components may be selectively included. The operation of each component will be described in detail below.

The next-day mission generation unit 310 may generate a mission for the next-day victory in the user terminal 110. Here, the victory may correspond to the next-day standard weight loss, for example, a target weight loss to be achieved by the next day. In addition, the mission may correspond to an exercise program for achieving the target weight loss of the user, and may be determined based on the game level of the user, for example. The next-day mission generation unit 310 may provide at least one mission for the target weight loss based on the current weight and current game level of the user. In one embodiment, the next-day mission generation unit 310 may classify the user into underweight, normal, obese, and severely obese and assign the game level according to the weight. Here, the next-day mission generation unit 310 may provide exercise programs such as running, squats, or the like according to the game level assigned to the user, but is not necessarily limited thereto, and may assign the target weight loss or target calorie consumption according to the game level of the user.

In one embodiment, the next-day mission generation unit 310 may determine a baseline weight loss through an artificial intelligence weight loss model that trains the exercise amount of the user as input data and the weight change amount of the user as output data. Here, the artificial intelligence weight loss model may correspond to an artificial intelligence algorithm that trains the past exercise pattern of the user and weight change data and models the correlation between the exercise amount and weight loss. The next-day mission generation unit 310 may receive the current weight and exercise amount of the user from the user terminal 110 and output the weight change amount of the user according to the exercise amount of the user through the artificial intelligence weight loss model. Here, the next-day mission generation unit 310 may receive exercise amount data including the number of steps, distance run, calorie consumption, and exercise time of the user, and set a standard weight loss goal suitable for the user through the artificial intelligence weight loss model.

In one embodiment, the next-day mission generation unit 310 may provide the usual behavioral data of the user to the artificial intelligence action plan model to determine the behavioral pattern of the user equal to or more than a certain standard frequency and determine at least one action plan based on the behavioral pattern. Here, the next-day mission generation unit 310 may be linked to the user terminal 110 to collect the usual behavioral data of the user through smartphone sensors such as an accelerometer, GPS, and gyroscope included in the user terminal 110. The next-day mission generation unit 310 may collect the usual behavioral data of the user, including exercise, walking, food intake, rest time, or the like that the user routinely performs, through the user terminal 110 and provide the data as input to the artificial intelligence action plan model. Here, the artificial intelligence action plan model may correspond to an artificial intelligence model that analyzes the usual behavioral data of the user and generates a user-customized action plan (that is, exercise program) for weight management.

In one embodiment, the next-day mission generation unit 310 may analyze the usual behavior data of the user based on the artificial intelligence action plan model to determine the behavior pattern based on the regularity of a specific behavior that is frequently repeated. Here, the next-day mission generation unit 310 may determine the behavior pattern based on the usual behavior data of the user that is repeated at a specific time period. For example, the next-day mission generation unit 310 may determine the behavior pattern by dividing the usual behavior data of the user into weekdays and weekends, but is not necessarily limited thereto and may determine the behavior pattern based on usual behavior data according to morning, lunch, and dinner time periods.

In one embodiment, the next-day mission generation unit 310 may determine the behavioral pattern of the user based on the artificial intelligence action plan model and provide at least one action plan based on the behavioral pattern to the user terminal 110. For example, when the behavioral pattern of the user is concentrated in the morning, the next-day mission generation unit 310 may provide the action plan, such as running, that may be performed in the morning. In addition, the next-day mission generation unit 310 is not necessarily limited thereto, and may provide a social action plan, such as “going hiking with other users”, or an alternative action plan, such as “eat fruit instead of a late-night snack”, based on the activity pattern of the user.

In one embodiment, the next-day mission generation unit 310 may analyze daily behavior data from the usual behavior data through the artificial intelligence action plan model, assign attention between the daily behavior data to determine a major behavior pattern, virtually generate major actions with the major behavior patterns, and determine at least one action plan from among the N (where N is a natural number) most similar action plans among all action plans. Here, the daily behavior data may correspond to daily behaviors performed by the user during the day, such as exercise, eating, and sleeping. The next-day mission generation unit 310 may assign an attention weight to each daily behavior data by analyzing the correlation between the daily behavior data based on the artificial intelligence action plan model. For example, the next-day mission generation unit 310 may assign a higher attention weight to daily behavior data with a higher correlation to exercise time, thereby determining the daily behavior data as the major behavior pattern.

In one embodiment, the next-day mission generation unit 310 may generate a virtual main action based on the main behavioral pattern of the user and provide N action plans similar to the virtual main action to the user terminal 110. Through this, the next-day mission generation unit 310 may provide a personalized, customized action plan to the user by providing an action plan similar to the usual behavioral data of the user. For example, when the user mainly exercises in the morning, the next-day mission generation unit 310 may generate a virtual main action such as “walk for 30 minutes at 7 a.m. tomorrow” and provide N action plans similar thereto (for example, go jogging at 7 a.m., ride a bicycle at 7 a.m., or the like).

The lifestyle pattern data collection unit 320 may collect the lifestyle pattern data of the user through the user terminal 110. Here, the lifestyle pattern data may correspond to data related to the daily activities of the user, and may further include, for example, the exercise, sleep, meal, activity level, and location data of the user. The lifestyle pattern data collection unit 320 may collect the lifestyle pattern data of the user, including exercise, sleep, meal, and activity level, through smartphone sensors such as GPS, accelerometer, and heart rate monitor installed in the user terminal 110, and store the collected data in the database 150. Here, the lifestyle pattern data collection unit 320 may calculate the daily calorie consumption from the collected user lifestyle pattern data and update the calculated daily calorie consumption in real time.

The victory achievement prediction unit 330 may analyze the lifestyle pattern data of the user to determine the past day with the most similar past lifestyle pattern data, and may predict whether or not victory is achieved based on the lifestyle pattern data of the past day. For example, the victory achievement prediction unit 330 may analyze the lifestyle pattern data of the user and compare the lifestyle pattern data with the past lifestyle pattern data including exercise, meals, and sleep recorded by the user in the past. Here, the victory achievement prediction unit 330 may determine the past day that is most similar to the lifestyle pattern data of the user based on criteria such as the exercise amount, eating habits, and sleeping patterns. In one embodiment, the victory achievement prediction unit 330 may determine whether or not victory is achieved based on the lifestyle pattern data of the pass day similar to the lifestyle pattern data of the user, and may predict the current possibility of victory of the user based on this. However, the present disclosure is not limited thereto, and the victory achievement prediction unit may calculate the current probability of victory.

In one embodiment, the victory achievement prediction unit 330 may determine a past population based on the location of the user, extract time-based behavioral feature data based on the lifestyle pattern data of the user, and determine a past day from the past population. Here, the time-based behavioral feature data may correspond to data that specifies the daily activities of the user by time zone, and may correspond to, for example, patterns such as exercise amount, calorie intake, and sleep time segmented by specific time zone. The victory achievement prediction unit 330 may determine the past population that includes the lifestyle pattern data of users who performed the same activity in the past at the corresponding location based on the location data of the user. For example, the victory achievement prediction unit 330 may generate the data on other users who exercised in the park as a single past population based on location data from the park where the user frequently exercises. In one embodiment, the victory achievement prediction unit 330 may derive a past day that is most similar to the current lifestyle pattern data of the user from the past population, and predict whether the user achieves victory based on whether or not the user achieves victory on that day.

The mission performance detection unit 340 may analyze the lifestyle pattern data of the user to determine whether to perform the mission when the victory is predicted. Here, the mission performance detection unit 340 may provide at least one action plan to the user terminal 110 and provide the victory achievement probability for each action plan. In one embodiment, the mission performance detection unit 340 may receive at least one action plan from the user terminal 110 and request the user to perform the mission based on the action plan. Here, the mission performance detection unit 340 may monitor the actual activity of the user by linking with the user terminal 110 and analyze the lifestyle pattern data of the user by time zone to calculate the exercise amount data.

In one embodiment, the mission performance detection unit 340 may additionally provide the user terminal 110 with a mission that can be executed during the remaining day when the predicted probability of achieving victory gradually decreases. Here, the mission performance detection unit 340 may monitor the lifestyle pattern data of the user through the user terminal 110 and, when the user's probability of achieving victory gradually decreases over time, may additionally provide the user with a new mission that can be executed during the remaining time. That is, the mission performance detection unit 340 may monitor the lifestyle pattern data of the user and calculate the exercise amount data of the user, and, when the probability of achieving the target weight loss or exercise amount is low, may provide an additional action plan, thereby improving the probability of winning.

The action plan change unit 350 may change at least one action plan based on the lifestyle pattern data of the past day when the victory is not predicted. Here, the action plan change unit 350 may predict the probability of victory by monitoring the current daily life pattern and weight change trend of the user in real time, and may adjust the action plan of the user when the victory is not predicted. For example, the action plan change unit 350 may change at least one of the action plans of the user to a high-intensity action plan including high-intensity interval training, when the victory is not predicted. In one embodiment, the action plan change unit 350 may change the action plan based on lifestyle pattern data of the past day that achieved victory among lifestyle pattern data of the past day. For example, the action plan change unit 350 may provide an optimized action plan to the user by referring to the exercise habits, eating habits, and activity level of the corresponding day based on the lifestyle pattern data of the past day when victory was achieved.

In one embodiment, the action plan change unit 350 may present the lifestyle habit content required in the future or the degree of increase of the action plan required in the future based on the lifestyle pattern data of the past day, through the user terminal 110. Here, the action plan change unit 350 may analyze the past lifestyle pattern data of the user, including the exercise amount, eating habits, activity time zone, sleep pattern, or the like, and may suggest a modified action plan based on behaviors that were effective in the past when the possibility of achieving the current goal has decreased. For example, when it is determined that the user is likely to fail to achieve the target exercise amount, the action plan change unit 350 may lower the exercise intensity, increase the exercise time, or suggest a different form of high-intensity exercise. In addition, the action plan change unit 350 may suggest a method of limiting the calorie intake of the user by analyzing the past lifestyle pattern data of the user and adding a meal control mission.

The user level determination unit 360 may analyze whether the victory or mission has been achieved and adjust the game level of the user or at least one action plan. Here, the user level determination unit 360 may adjust the game level of the user or action plan by comprehensively analyzing whether the user has achieved the baseline weight loss of the next day or has achieved the action plan, such as the activity level, calorie consumption, and improvement of lifestyle habits of the user. For example, when the user has won or achieved the mission, the user level determination unit 360 may raise the game level and provide an action plan with a higher difficulty level. In addition, when the user has not won or achieved the mission, the user level determination unit 360 may lower the game level and provide the action plan with an easier difficulty level, thereby suggesting a personalized action plan, such as lowering the exercise intensity or changing the meal plan.

In one embodiment, the user level determination unit 360 may determine level up or level down based on the mission achievement win rate, victory achievement win rate, and weight change between the same day and the next day of the user. Here, the user level determination unit 360 may record the victory when the user achieves the baseline weight loss amount for the next day and calculate the victory achievement win rate. In addition, the user level determination unit 360 may record the mission as a success when the user successfully achieves the action plan and may accumulate the mission achievement record to calculate the mission achievement win rate. The user level determination unit 360 may comprehensively analyze the mission achievement win rate, victory achievement win rate, and weight change amount of the user to adjust the game level of the user up or down.

For example, the user level determination unit 360 may increase the game level of a specific user by granting him/her a mission of higher difficulty when the user achieves consecutive missions, wins, and weight loss successes. Conversely, the user level determination unit 360 may lower the level of a specific user and suggest a personalized action plan when the user repeatedly fails the mission or the weight change stagnates.

The control unit 370 may control the overall operation of the smart weight management device 130 and manage the control flow or data flow between the next day mission generation unit 310, the lifestyle pattern data collection unit 320, the victory achievement prediction unit 330, the mission performance detection unit 340, the action plan change unit 350, and the user level determination unit 360.

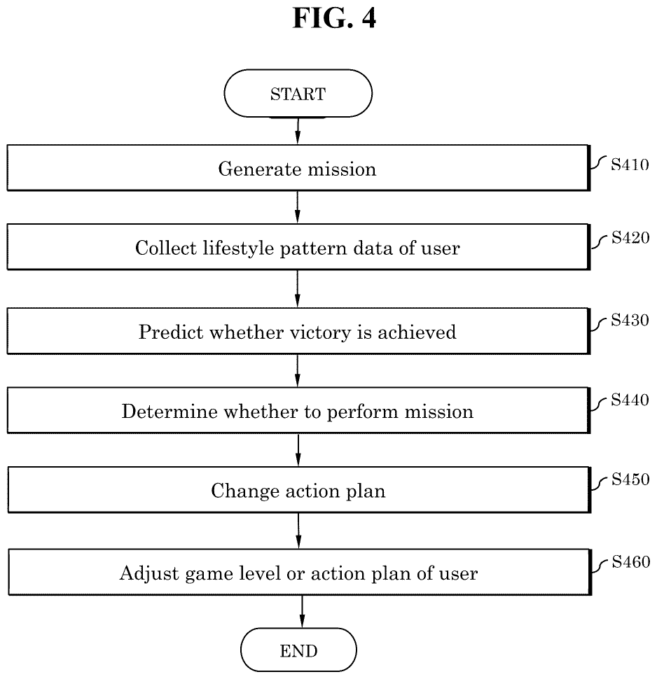

FIG. 4 is a flowchart illustrating one embodiment of a transformer-based electronic disclosure document service platform method according to the present disclosure.

Referring to FIG. 4, the smart weight management device 130 may generate the mission for the victory of the next day in the user terminal 110 through the next day mission generation unit 310 (Step S410). Here, the smart weight management device 130 may determine the baseline weight loss through the artificial intelligence weight loss model that trains the exercise amount of the user as the input data and the weight change of the user as the output data.

In addition, the smart weight management device 130 may collect the lifestyle pattern data of the user through the user terminal 110 based on the lifestyle pattern data collection unit 320 (Step S420). The smart weight management device 130 may analyze the lifestyle pattern data of the user based on the victory achievement prediction unit 330 to determine the past day with the most similar past lifestyle pattern data and predict whether victory is achieved based on the lifestyle pattern data of the past day (Step S430).

The smart weight management device 130 may analyze the lifestyle pattern data of the user to determine whether to perform the mission when the achievement of victory is predicted through the mission performance detection unit 340 (Step S440). When the achievement of victory is not predicted through the action plan change unit 350, the smart weight management device 130 may change at least one action plan based on the lifestyle pattern data of the past day (Step S450). The smart weight management device 130 may analyze whether the victory or mission achievement is predicted through the user level determination unit 360 to adjust the game level of the user or at least one action plan (Step S460).

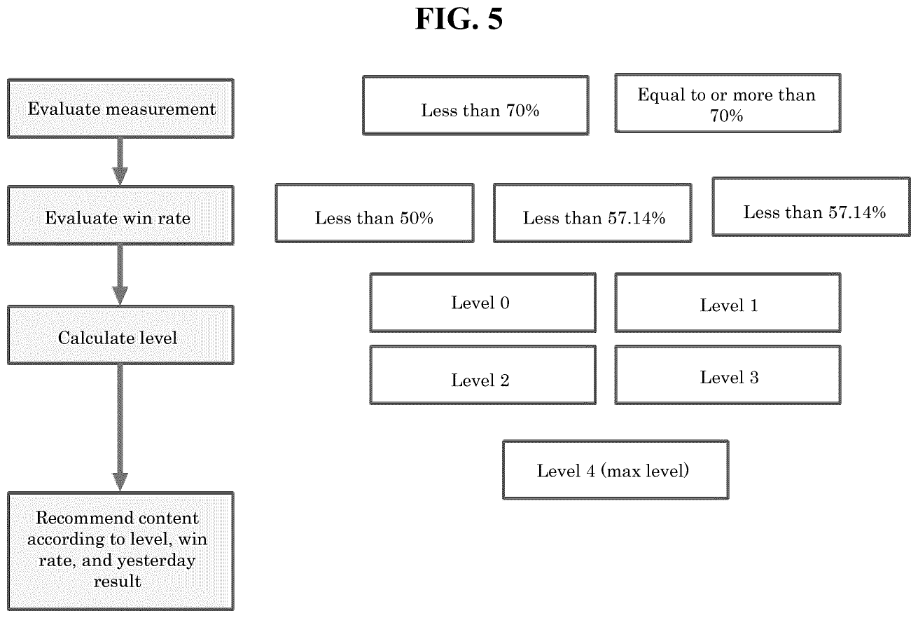

FIG. 5 is a diagram illustrating a user level determination process according to mission performance according to one embodiment of a smart weight management device according to the present disclosure.

Referring to FIG. 5, the smart weight management device 130 may assign a basic game level to a new user via the user terminal 110, and may provide the user with promotion conditions for advancing from the basic game level to the next level of game level depending on whether the usual behavioral data of the user is measured. For example, the smart weight management device 130 may grant the basic game level to the new user and, may grant the user the next level of game level when the measurement rate of the usual behavioral data of the user is 70% or higher for a week.

In one embodiment, the smart weight management device 130 may grant the user the next level of game level based on the mission achievement win rate and victory achievement win rate of the user. For example, when the win rate of the user is 50% or higher on a weekly basis, the smart weight management device 130 may grant the user the next level of game level and provide a mission with a higher difficulty level. The smart weight management device 130 is not necessarily limited thereto, and may perform the game level promotion process based on the mission achievement win rate and victory achievement win rate of the user according to a standard value (for example, 50% or higher) set in the user terminal 110.

In one embodiment, the smart weight management device 130 may provide content based on the game level and win rate of the user, and whether or not the user achieved victory as of yesterday. Here, the content may correspond to the cause of defeat and victory, and may not necessarily be limited thereto, and may correspond to visual effects based on defeat and victory. For example, when the user loses, the smart weight management device 130 may provide effect content related to the weight gain of the user to the user terminal 110. However, the smart weight management device may not necessarily be limited thereto, and may provide graphic content related to the cause of the user's defeat, including late-night snacks, drinking, overeating, and carbohydrate intake. In addition, when the user wins, the smart weight management device 130 may provide effect content related to the user's weight loss, and may not necessarily be limited thereto, and may provide the user's cause of victory, including the number of steps, sleeping time, late-night snacks, drinking, overeating, and carbohydrate intake, to the user terminal 110.

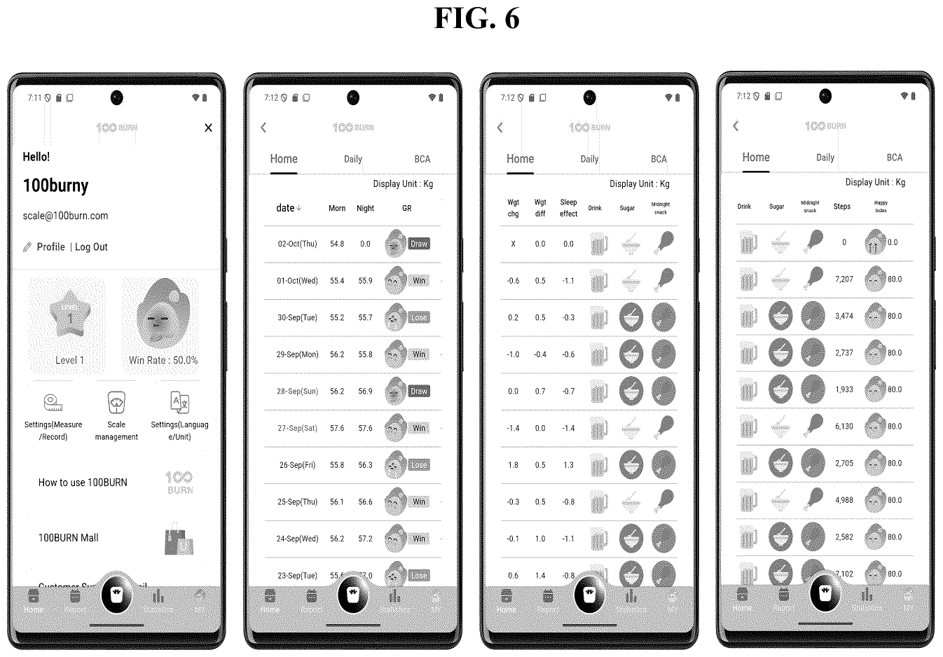

FIG. 6 is a diagram illustrating one embodiment of the smart weight management device according to the present disclosure.

Referring to FIG. 6, the smart weight management device 130 may provide a profile including an ID and nickname for a specific user. Here, the smart weight management device 130 may provide the user terminal 110 with the level and win rate for the specific user, and is not necessarily limited thereto. The smart weight management device 130 may also provide the user terminal 110 with information regarding the usual behavioral data and daily weight change trends of the user.

In one embodiment, the smart weight management device 130 may provide the user terminal 110 with the daily win, draw, and loss results for a specific user. Here, the smart weight management device 130 may provide the user with the user's morning and nighttime weight, and may also adjust the game level or action plan of the user by comprehensively analyzing whether the user has achieved the baseline weight loss of the next day or has achieved the action plan, such as improving activity levels, calorie consumption, and lifestyle habits of the user.

In one embodiment, the smart weight management device 130 may provide the user terminal 110 with the weight change, sleep effect, drinking status, meal type, and late-night snack consumption of the user based on the win or loss result of the user. For example, when the user loses, the smart weight management device 130 may provide the user terminal 110 with effect content related to the weight gain of the user, and may provide the user terminal 110 with reasons for the failure, such as sleep effect, drinking status, meal type, and late-night snack consumption.

In one embodiment, the smart weight management device 130 may calculate the exercise amount and happiness index of the user and provide them to the user terminal 110. Here, the happiness index may correspond to the satisfaction level of the user based on whether the user achieved victory. In one embodiment, the smart weight management device 130 may provide the exercise amount and happiness index of the user to the user terminal 110 in real time, thereby allowing the user to intuitively check his/her physical activity and emotional state and focus on his/her weight management goals.

Although the present disclosure has been described above with reference to preferred embodiments thereof, it will be understood by those skilled in the art that various modifications and changes may be made to the present disclosure without departing from the spirit and scope of the present disclosure as set forth in the claims below.

[Detailed Description of Main Elements]

| | |
|---|---|
| 100: smart weight management | |
| system | |
| 110: user terminal | |
| 130: smart weight management | |
| device | |
| 150: database | |
| 210: processor | 230: memory |
| 250: user input/output unit | 270: network input/output unit |
| 290: communication port unit | |
| 310: next-day mission generation | 320: lifestyle pattern data |
| unit | collection unit |
| 330: victory achievement | 340: mission performance |
| prediction unit | detection unit |
| 350: action plan change unit | 360: user level determination unit |
| 370: control unit | |

1. A gamification-based smart weight management device comprising:
a next-day mission generation unit that generates a mission for victory of a next day on a user terminal, the victory representing a baseline weight loss for the next day and the mission being determined based on a game level of a user and including at least one action plan;
a lifestyle pattern data collection unit that collects lifestyle pattern data of the user through the user terminal;
a victory achievement prediction unit that analyzes the lifestyle pattern data of the user to determine a past day with a most similar lifestyle pattern data and predicts whether the victory is achieved based on a lifestyle pattern data from the past day;
a mission performance detection unit that analyzes the lifestyle pattern data of the user to determine whether the mission is performed when the victory is predicted;
an action plan change unit that changes the at least one action plan based on lifestyle pattern data of the past day when the victory is not predicted; and
a user level determination unit that analyzes whether the victory or the mission is achieved and adjusts the game level of the user or the at least one action plan.

2. The gamification-based smart weight management device of claim 1, wherein the next-day mission generation unit determines the baseline weight loss through an artificial intelligence weight loss model that trains an exercise amount of the user as input data and a weight change of the user as output data.

3. The gamification-based smart weight management device of claim 1, wherein the next-day mission generation unit provides usual behavioral data of the user to an artificial intelligence action plan model to determine a behavioral pattern of the user equal to or more than a certain standard frequency and determine the at least one action plan based on the behavioral pattern.

4. The gamification-based smart weight management device of claim 1, wherein the next-day mission generation unit analyzes daily behavior data from the usual behavior data through the artificial intelligence action plan model, assigns attention between the daily behavior data to determine a major behavior pattern, virtually generates a major action as the major behavior pattern, and determines the most similar N (where N is a natural number) action plans among all action plans as at least one action plan.

5. The gamification-based smart weight management device of claim 1, wherein the victory achievement prediction unit determines past population based on a location of the user, extracts time-based behavioral feature data based on the lifestyle pattern data of the user, and determines the past day from the past population.

6. The gamification-based smart weight management device of claim 1, wherein the mission performance detection unit additionally provides the user terminal with a mission that can be executed in the remaining day when a predicted probability of achieving the victory gradually decreases.

7. The gamification-based smart weight management device of claim 1, wherein the action plan change unit presents lifestyle habit content required in the future or a degree of increase in an action plan required in the future based on the lifestyle pattern data of the past day, through the user terminal.

8. The gamification-based smart weight management device of claim 1, wherein the user level determination unit determines level up or level down based on a mission achievement win rate, victory achievement win rate, and weight change between the same day and the next day of the user.
